(19) United States
(12) Patent Application Publication
(10) Pub. No.: US 2022/0080044 A1
(43) Pub. Date: Mar. 17, 2022

(54) METHOD FOR PREVENTING OR TREATING PERIPHERAL ARTERIAL OCCLUSIVE DISEASE

(71) Applicant: National Yang-Ming University, Taipei (TW)

(72) Inventors: Jaw-Wen Chen, Taipei (TW); Ting-Ting Chang, Taipei (TW)

(21) Appl. No.: 17/019,417

(22) Filed: Sep. 14, 2020

(51) Int. Cl. A61K 39/395; A61K 31/513; A61K 31/18; A61K 31/17; A61K 31/4192; A61P 17/02; A61P 9/10

(52) U.S. Cl. CPC A61K 39/3955; A61K 31/513; A61K 31/18; A61P 9/10; A61K 31/4192; A61P 17/02; A61K 31/17

(57) ABSTRACT

Provided is a method for preventing or treating a peripheral arterial occlusive disease (PAOD), including administering to a subject a CXC chemokine ligand 5 (CXCL5) antagonist in an effective amount. Also provided is a method for preventing or treating a peripheral ischemic tissue or a tissue damaged by peripheral ischemia through inhibition of CXCL5 to enhance angiogenesis, which may lead to an acceleration of wound healing.

### BACKGROUND

### 1. Technical Field

The present disclosure relates to methods for improving a peripheral arterial occlusive disease (PAOD), and particularly to methods for preventing or treating peripheral ischemia.

### 2. Description of Related Art

Peripheral arterial occlusive disease (PAOD) is a common circulatory problem involving blockage in arteries that reduces blood flow to limbs. PAOD may cause the extremities to have inadequate blood flow and result in symptoms such as intermittent claudication, which reduces patient's mobility.

PAOD patients, such as those suffering from diabetes, experience abnormalities in the blood vessels that supply blood to the skin. Consequently, these patients may further experience ulcerations or even have areas of necrosis (i.e., tissue death) on certain parts of their skin. Ischemic lesions are extremely painful and debilitating, and heal slowly and tend to occur on hands and fingers, e.g., knuckles, or other bony prominences, such as elbows, knees, hips, ankles and toes. Therefore, problems resulted from PAOD that affect the ambulatory nature of patients are important in view of physical risk. This may also convey an emotional risk, as these problems significantly disrupt the fundamental independence of patients by limiting their ability to walk.

Diabetic patients usually have impaired wound healing, and 15% of the population with diabetes are expected to develop into foot ulcers during their lifetime. These ulcers tend to be chronic in nature, as they do not heal or heal extremely slow. Diabetic foot ulcers are a serious problem for diabetic patients, as up to 25% of diabetic foot ulcers would eventually require amputation due to peripheral vascular lesions.

The promotion of angiogenesis or neovascularization is one of the strategies for improving PAOD. However, diabetic vasculopathy also accompanies systemic vascular inflammation.

Accordingly, the therapeutic efficacy in diabetic vasculopathy cannot be comparable to that in arteriosclerosis associated with cardiovascular disease because there is no effective treatment so far for controlling inflammation caused by diabetic vasculopathy.

Hence, there is still an unmet need for improved prevention or treatment of PAOD conditions, e.g., limb ischemia.

### SUMMARY

In view of the foregoing, the present disclosure provides a method for preventing or treating a condition or disorder susceptible to amelioration by stimulating angiogenesis through the inhibition of CXC chemokine ligand 5 (CXCL5).

In one embodiment of the present disclosure, a method for preventing or treating a PAOD in a subject in need thereof is provided. The method comprising administering to the subject an effective amount of a CXCL5 antagonist.

In one embodiment of the present disclosure, the CXCL5 antagonist is capable of inhibiting CXCL5 activity by preventing the binding of CXCL5 to its receptor. In another embodiment, the CXCL5 antagonist is an antibody or an aptamer directed against CXCL5 or the receptor of CXCL5. In yet another embodiment, the CXCL5 antagonist may be an anti-CXCL5 antibody or a fragment thereof, a soluble form of CXC chemokine receptor 2 (CXCR2), a CXCR2 blocker, a soluble form of CXCR1, a CXCR1 blocker, a soluble form of Duffy antigen receptor for chemokine (DARC), or a DARC blocker. In yet further another embodiment, the CXCL5 antagonist is selected from the group consisting of a CXCL5 neutralizing antibody, AZD5069 (i.e., N-[2-[(2,3-difluorophenyl)methylsulfanyl]-6-[(2R,3S)-3,4-dihydroxybutan-2-yl]oxypyrimidin-4-yl]azetidine-1-sulfonamide), reparixin, SB225002 (i.e., N-(2-hydroxy-4-nitrophenyl)-N′-(2-bromo-phenyl)-urea), SB265610 (i.e., N-(2-bromophenyl)-N′-(7-cyano-1H-benzotriazol-4-yl)urea), and a combination thereof.

In one embodiment of the present disclosure, the PAOD to be prevented or treated by the method may be limb ischemia, diabetic ulcer, gangrene, intermittent claudication, Buerger's syndrome, Raynaud's syndrome, or vasculitis. In another embodiment, the diabetic ulcer is a diabetic foot ulcer. In yet another embodiment, the limb ischemia is chronic limb ischemia.

In one embodiment of the present disclosure, a method for preventing or treating a peripheral ischemic tissue or a tissue damaged by peripheral ischemia in a subject in need thereof is also provided. The method comprises administering to the subject a pharmaceutical composition comprising the CXCL5 antagonist in an effective amount to induce angiogenesis in the subject, and a pharmaceutically acceptable carrier thereof.

In one embodiment of the present disclosure, the peripheral ischemic tissue or the peripheral ischemia may be caused by at least one of diabetes, chronic artery occlusion, Buerger's disease, Raynaud's disease, vascular spasm, scleroderma, and vasculitis. In another embodiment, the peripheral ischemic tissue or the tissue damaged by peripheral ischemia comprises a chronic wound, a digital ischemic lesion, a digital ulcer, or a digital necrotic lesion.

In one embodiment of the present disclosure, the administration of the CXCL5 antagonist results in stimulation of angiogenesis in the subject. In another embodiment, the administration promotes healing of the ischemic tissue or accelerates wound healing in the subject. In still another embodiment, the administration promotes the re-epithelialization or the matrix deposition in the ischemic tissue or the damaged tissue. In yet another embodiment, the administration reduces the rest pain associated with the peripheral ischemic tissue or the peripheral ischemia. In yet another further embodiment, the administration reduces the development of a new peripheral ischemic tissue in the subject.

In one embodiment of the present disclosure, the effective amount of the CXCL5 antagonist is from about 0.01 mg/kg to about 100 mg/kg, such as from about 0.1 mg/kg to about 80 mg/kg, from about 0.5 mg/kg to about 70 mg/kg, from about 1 mg/kg to about 60 mg/kg, from about 5 mg/kg to about 50 mg/kg, from about 10 mg/kg to about 40 mg/kg, and from about 15 mg/kg to about 30 mg/kg.

In one embodiment of the present disclosure, the CXCL5 antagonist is administered orally, intraperitoneally, intravenously, intradermally, intramuscularly, subcutaneously, or transdermally.

In one embodiment of the present disclosure, the CXCL5 antagonist is administered 1 to 2 times over a period of 2 to 4 days. In another embodiment, the CXCL5 antagonist is administered 8 to 15 times over a period of 3 to 5 weeks.

In the present disclosure, by using the CXCL5 antagonist, the method provided in the present disclosure may improve the functions of endothelial progenitor cells (EPCs) and human aortic endothelial cells (HAECs), so as to enhance angiogenesis. Hence, the method of the present disclosure is effective in improving the healing ability of wound and ischemia, as well as reducing rest pain associated with the ischemia and preventing the development of a new ischemic tissue. The method of using a CXCL5 antagonist of the present discourse is useful in accelerating angiogenic process, and thus effective in treating PAOD and improving peripheral ischemia.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The present disclosure can be more fully understood by reading the following descriptions of the embodiments, with reference made to the accompanying drawings.

FIGS. 1A to 1D are graphs illustrating the levels of CXCL5 in plasma (n=6; FIG. 1A) and in supernatants (n=6; FIG. 1B) from mononuclear cells, early endothelial progenitor cells (EPCs) (n=6; FIG. 1C), and late EPCs (n=6; FIG. 1D) of the normal and diabetic subjects. “N” represents cells cultured from “n” different individuals, and cells cultured from each individual are experimented for three independent experiments. DM: diabetes mellitus. * P<0.05, ** P<0.01.

FIGS. 2A to 2H are graphs illustrating the effect of different amounts of CXCL5 neutralizing antibody on the angiogenesis and migration abilities of EPCs. FIGS. 2A to 2D show the tube formation and migration of late EPCs from the diabetic subjects induced by the treatment of CXCL5 neutralizing antibody and the percentages thereof relative to normal subjects (n=3). FIGS. 2E to 2H show the tube formation and migration of high glucose-stimulated late EPCs from the normal subjects induced by the treatment of CXCL5 neutralizing antibody and the percentages thereof relative to unstimulated EPCs (n=3). “N” represents cells cultured from “n” different individuals, and cells cultured from each individual are experimented for three independent experiments. DM: diabetes mellitus. CXCL5 mAb: CXCL5 neutralizing antibody. Control: late EPCs from the normal subjects without high glucose stimulation. HG: high glucose (25 mM). * P<0.05, ** P<0.01.

FIGS. 3A to 3F are graphs illustrating the effect of different amounts of CXCL5 neutralizing antibody on the vascular endothelial growth factor (VEGF) and stromal cell-derived factor 1 (SDF-1) expressions in late EPCs from the diabetic subjects (n=3; FIGS. 3A to 3C) and in high glucose-stimulated late from the normal subjects (n=3; FIGS. 3D to 3F). “N” represents cells cultured from “n” different individuals, and cells cultured from each individual are experimented for three independent experiments. DM: diabetes mellitus. CXCL5 mAb: CXCL5 neutralizing antibody. Control: late EPCs from the normal subjects without high glucose stimulation. HG: high glucose (25 mM). * P<0.05, ** P<0.01.

FIGS. 4A to 4G are graphs illustrating the effect of different amounts of CXCL5 neutralizing antibody on the angiogenesis and migration abilities (n=3; FIGS. 4A to 4D) and the VEGF and SDF-1 expressions (n=3; FIGS. 4E to 4G) in high glucose-stimulated human aortic endothelial cells (HAECs). CXCL5 mAb: CXCL5 neutralizing antibody. Control: HAECs without high glucose stimulation. HG: high glucose (25 mM). * P<0.05, ** P<0.01.

FIGS. 5A and 5B are graphs illustrating the foot blood flow monitored by laser Doppler imaging system in each group of mice (non-DM group, n=6; DM, n=8; DM+IgG 10 μg group, n=6; DM+IgG 100 μg group, n=6; DM+CXCL5 10 μg mAb group, n=6; DM+CXCL5 100 mAb group, n=6). FIG. 5A shows representative evaluation of the ischemic (right) and non-ischemic (left) hindlimbs which is performed before, immediately after, and 4 weeks after the hindlimb ischemia surgery. FIG. 5B shows quantitative evaluation of blood flow expressed as a ratio of blood flow in ischemic limb to that in non-ischemic one. DM: diabetes mellitus. CXCL5 mAb: CXCL5 neutralizing antibody. * P<0.05, ** P<0.01.

FIG. 6 is a graph illustrating the levels of the circulating EPCs in each group of mice (non-DM group, n=6; DM, n=8; DM+IgG 10 μg group, n=6; DM+IgG 100 μg group, n=6; DM+CXCL5 10 μg mAb group, n=6; DM+CXCL5 100 μg mAb group, n=6), measured by flow cytometry before and after the hindlimb ischemia surgery. DM: diabetes mellitus. CXCL5 mAb: CXCL5 neutralizing antibody. OP: operation of hindlimb ischemia. * P<0.05, ** P<0.01.

FIGS. 7A to 7C are graphs illustrating the VEGF and SDF-1 expressions in thigh muscles of DM mice treated with CXCL5 100 μg mAb, n=3, measured by Western blotting after the hindlimb ischemia surgery. DM: diabetes mellitus. CXCL5 mAb: CXCL5 neutralizing antibody. * P<0.05, ** P<0.01.

FIGS. 8A to 8E are graphs illustrating the effect of CXCL5 neutralizing antibody on wound healing in the diabetic mice (non-DM group, n=24; DM, n=20; DM+IgG 10 μg group, n=12; DM+IgG 100 μg group, n=12; DM+CXCL5 10 μg mAb group, n=12; DM+CXCL5 100 μg mAb group, n=12). FIGS. 8A and 8B show the representative photographs of wound healing in each group of mice over a time period and the statistical analyses of a ratio of wound area (%) to the initial area on day 0 measured over time. FIGS. 8C to 8E show H&E-stained, anti-CD31 immunostaining and Masson's trichrome staining sections of the subcutaneous tissue observed using a light microscope, respectively. DM: diabetes mellitus. CXCL5 mAb: CXCL5 neutralizing antibody. * P<0.05, ** P<0.01.

FIGS. 9A and 9B are graphs illustrating the effect of CXCL5 neutralizing antibody on aortic sprouting ex vivo (non-DM group, n=10; DM, n=12; DM+IgG 10 μg group, n=6; DM+IgG 100 μg group, n=6; DM+CXCL5 10 μg mAb group, n=6; DM+CXCL5 100 μg mAb group, n=6). DM: diabetes mellitus. CXCL5 mAb: CXCL5 neutralizing antibody. ** P<0.01.

FIGS. 10A and 10D are graphs illustrating the effect of CXCL5 neutralizing antibody on matrigel plug neovascularization in vivo (non-DM group, n=10; DM, n=12; DM+IgG 10 μg group, n=6; DM+IgG 100 μg group, n=6; DM+CXCL5 10 μg mAb group, n=6; DM+CXCL5 100 μg mAb group, n=6). FIGS. 10A and 10B show the matrigel plugs in the mice of each group and the quantitative analysis of neovascularization in matrigel plugs measured by hemoglobin contents. FIGS. 10C and 10D show the H&E-stained and anti-CD31 immunostaining sections of the matrigel plugs, respectively. DM: diabetes mellitus. CXCL5 mAb: CXCL5 neutralizing antibody. ** P<0.01.

### DETAILED DESCRIPTION OF THE EMBODIMENTS

The following examples are used for illustrating the present disclosure. A person skilled in the art can easily conceive the other advantages and effects of the present disclosure, based on the disclosure of the specification. The present disclosure can also be implemented or applied as described in different examples. It is possible to modify or alter the following examples for carrying out this disclosure without contravening its spirit and scope, for different aspects and applications.

It is further noted that, as used in this disclosure, the singular forms “a,” “an,” and “the” include plural referents, unless expressly and unequivocally limited to one referent. The term “or” is used interchangeably with the term “and/or,” unless the context clearly indicates otherwise.

As used herein, the term “comprising” or “comprises” is used in reference to compositions, methods, and respective component(s) thereof, which are essential to the present disclosure, yet open to the inclusion of unspecified elements, whether essential or not.

The present disclosure is directed to a method for preventing or treating a peripheral arterial occlusive disease (PAOD) in a subject in need thereof. The method comprises administering to the subject an effective amount of a CXCL5 antagonist, wherein the CXCL5 antagonist is capable of inhibiting CXCL5 activity.

The present disclosure is also directed to a method for preventing or treating a peripheral ischemic tissue or a tissue damaged by peripheral ischemia in a subject in need thereof, comprising administering to the subject an effective amount of the CXCL5 antagonist, wherein the CXCL5 antagonist is capable of stimulating angiogenesis in the subject.

In one embodiment of the present disclosure, the CXCL5 antagonist is capable of preventing the binding of CXCL5 to its receptor. In another embodiment, the CXCL5 antagonist is an agent that inhibits intracellular signaling generated on binding of CXCL5 to its receptor. For example, the CXCL5 antagonist may be directed against at least one of CXCL5 and the receptor of CXCL5, thereby blocking the CXCL5 signaling. As used herein, the receptor of CXCL5 includes, but is not limited to, CXCR2, CXCR1, and DARC [1-3].

As used herein, the terms “CXCR2” or “CXCR2 receptor” are used interchangeably and have their general meaning in the art. The CXCR2 receptor may be from any source, but typically is a mammalian (e.g., human or non-human primate) CXCR2 receptor. In one embodiment of the present disclosure, the CXCR2 receptor is a human CXCR2 receptor.

As used herein, the term “DARC,” also referred to as the Duffy blood group antigen, refers to a promiscuous receptor for several chemokines, which act as communication signals. The DARC binds to chemokines of both the CC and CXC classes, the melanoma growth stimulatory activity (MSGA-α/CXCL1), interleukin 8 (CXCL8), regulated upon activation normal T-expressed and secreted (RANTES/CCL5), monocyte chemotactic protein-1 (CCL2), neutrophil activating protein 2 and 3, growth-related gene alpha, CXCL5, and angiogenesis-related platelet factor 1.

As used herein, the term “CXCL5” has its general meaning in the art. CXCL5 is a natural ligand of the CXCR2 receptor, and may be from any source, but typically is a mammalian (e.g., human or non-human primate) CXCL5. In one embodiment of the present disclosure, the CXCL5 is a human CXCL5.

As used herein, the term “CXCL5 antagonist” includes any entity that, upon administration to a subject, results in inhibition or down-regulation of a biological activity associated with CXCL5 in the subject, including any of the downstream biological effects otherwise resulting from the binding of CXCL5 to its receptor. The CXCL5 antagonist includes any agent that may inhibit CXCL5 activity or block activation of the receptor of CXCL5, or any of the downstream biological effects of activation of the receptor of CXCL5. Such a CXCL5 antagonist includes any agent that is able to interact with CXCL5, so that its normal biological activity is prevented or reduced. For example, said agent may be a small organic molecule or an antibody directed against CXCL5, such as a CXCL5 neutralizing antibody, which can block the interaction between CXCL5 and its receptor, or which can block the activity of CXCL5. The CXCL5 antagonist may also be a small molecule or an antibody directed against the receptor of CXCL5, which may act by occupying the ligand binding site or a portion thereof of the receptor, thereby making the receptor inaccessible to its ligand, CXCL5.

In one embodiment of the present disclosure, the CXCL5 antagonist is an antibody or an aptamer directed against the receptor of CXCL5 or CXCL5. In another embodiment, the CXCL5 antagonist is an anti-CXCL5 antibody or a fragment thereof, a soluble form of CXCR2, a CXCR2 blocker, a soluble form of CXCR1, a CXCR1 blocker, a soluble form of DARC, or an DARC blocker. In still another embodiment, the CXCL5 antagonist is selected from the group consisting of a CXCL5 neutralizing antibody, AZD5069, reparixin, SB225002, SB265610, and a combination thereof.

As used herein, the term “aptamer” refers to a class of molecules that represents an alternative to antibodies in term of molecular recognition. The aptamer is an oligonucleotide or oligopeptide sequences with the capacity to recognize virtually any class of target molecules with high affinity and specificity.

In one embodiment of the present disclosure, the PAOD encompasses limb ischemia, diabetic ulcer, gangrene, intermittent claudication, Buerger's syndrome, Raynaud's syndrome and vasculitis. In another embodiment, the subject to be treated with the method of the present disclosure suffers from diabetes, chronic artery occlusion, vascular spasm, scleroderma or vasculitis. In yet another embodiment, the peripheral ischemic tissue or the peripheral ischemia is caused by at least one of diabetes, chronic artery occlusion, Buerger's disease, Raynaud's disease, vascular spasm, and scleroderma.

The limb ischemia comprises chronic limb ischemia and acute limb ischemia (ALI). Chronic limb ischemia progresses into critical limb ischemia (CLI) that leads to the distal limb at risk of amputation, and acute limb ischemia appears a rapid loss of blood flow that may damage tissues within hours. CLI often comes associated with diabetes, resulting in compromised vasculature, exaggerated tissue damage, and chronic ischemic rest pain. Further, Buerger's disease compromises blood flow to hands and feet, resulting eventually in the loss of fingers and toes. PAOD generally results in impaired wound healing, ulcers and tissue necrosis in limbs and extremities that may cause loss of the affected limb as a result of non-traumatic amputation.

The ankle brachial pressure index (ABPI) and the toe brachial pressure index (TBPI) are interpreted to give an indication of the status of the arterial system of the patient. Typical results are represented as below:

(1) ABPI>0.9 to 1.2: normal;

(2) ABPI=0.8 to 0.9: mild PAOD (which may present an inflow disease);

(3) ABPI=0.5 to 0.79: moderate ischemia/intermittent claudication (which would benefit from vascular surgeon for consulting to expedite wound healing);

(4) ABPI=0.35 to 0.49: moderately severe ischemia (which would be recommended for urgent vascular surgery consulting);

(5) ABPI=0.2 to 0.34: severe ischemia (which would be recommended for urgent vascular surgery consulting);

(6) ABPI<0.2: likely critical ischemia, in consideration of absolute pressure and clinical picture (which would be recommended for urgent vascular surgery consulting);

(7) TBPI>0.7: normal;

(8) TBPI=0.64 to 0.7: borderline; and

(9) TBPI<0.64: abnormal indication of PAOD (which would be recommended for urgent vascular surgery consulting).

In the PAOD patients, the numbers of endothelial progenitor cells (EPCs) are reduced, and the function of EPCs as defined by colony forming capacity and migratory activity is markedly reduced and associated with reduced neovascularization in hindlimb ischemia. Similarly, the numbers of EPCs are reduced in patients with type I or type II diabetes. The reduction of the numbers of EPCs and their function involve in some of the vascular complications, such as endothelial dysfunction, which predispose patients to the impaired neovascularization after ischemic events.

As used herein, the term “peripheral ischemic tissue” and the equivalents thereof refer to a tissue that has a decreased blood supply caused by any constriction, damage, or obstruction of the vasculature for supplying the peripheral tissue. As used herein, the term “tissue damaged by peripheral ischemia” and the equivalents thereof refer to morphological, physiological, and/or molecular damage to a tissue or cells as a result of a period of peripheral ischemia.

As used herein, the term “chronic wound” refers generally to a wound that has not healed within about three months, but can be wounds that have not healed within about one or two months. Chronic skin wounds include, for example, a diabetic ulcer, a venous ulcer, a trauma-induced ulcer, a pressure ulcer, a vasculitic ulcer, an arterial ulcer, a sickle cell ulcer, and mixed ulcers.

The methods and the CXCL5 antagonist of the present disclosure may be used to treat a variety of conditions that would benefit from stimulation of angiogenesis, stimulation of vasculogenesis, increased blood flow, and/or increased vascularity.

As used herein, the term “angiogenesis” indicates the growth or formation of blood vessels. Angiogenesis includes the growth of new blood vessels from pre-existing vessels, as well as vasculogenesis, which refers to spontaneous blood-vessel formation, and intussusception, which refers to new blood vessel formation by splitting off existing ones. Angiogenesis encompasses “neovascularization,” “regeneration of blood vessels,” “generation of new blood vessels,” and “revascularization.”

As used herein, the term “treating” or “treatment” refers to obtaining a desired pharmacologic and/or physiologic effect, e.g., stimulation of angiogenesis. The effect may be prophylactic in terms of completely or partially preventing a disease or symptom thereof or may be therapeutic in terms of completely or partially curing, alleviating, relieving, remedying, ameliorating a disease or an adverse effect attributable to the disease.

As used herein, the terms “symptoms associated with PAOD,” “symptoms resulting from ischemia,” and “symptoms caused by ischemia” refer to symptoms that include impaired, or loss of organ function, cramping, claudication, numbness, tingling, weakness, pain, reduced wound healing, inflammation, skin discoloration, and gangrene. “Treatment” as used herein covers any treatment of a disease and includes: (a) preventing a disease or condition (e.g., preventing the loss of a skin graft or a re-attached limb due to inadequate blood flow) from occurring in a subject who may be predisposed to the disease but has not yet been diagnosed as having it; (b) inhibiting the disease or symptom thereof, e.g., slowing down or arresting its development; or (c) relieving the disease or symptom thereof (e.g., enhancing the development of neovascularization around an ischemic tissue to improve blood flow to a tissue). In the context of the present disclosure, stimulation of angiogenesis is employed for a subject having a disease or condition amenable to treatment by increasing vascularity and increasing blood flow. Such a subject can be identified by a health care professional based on results from any suitable diagnostic method.

As used herein, the terms “patient” and “subject” are used interchangeably. The term “subject” means a human or animal. Examples of the subject include, but are not limited to, human, monkey, mice, rat, woodchuck, ferret, rabbit, hamster, cow, horse, pig, deer, dog, cat, fox, wolf, chicken, emu, ostrich, and fish. In certain embodiments of the present disclosure, the subject is a mammalian, e.g., a primate, such as a human.

As used herein, the phrase “an effective amount” refers to the amount of an active agent (e.g., CXCL5 antagonist) that is required to confer a desired therapeutic effect (e.g., a desired level of angiogenic stimulation) on the treated subject. Effective doses will vary, as recognized by those skilled in the art, depending on routes of administration, excipient usage, the possibility of co-usage with other therapeutic treatment, and the condition to be treated.

In one embodiment of the present disclosure, the effective amount of the CXCL5 antagonist is from about 0.01 mg/kg to about 100 mg/kg, such as from about 0.1 mg/kg to about 80 mg/kg, from about 0.5 mg/kg to about 70 mg/kg, from about 1 mg/kg to about 60 mg/kg, from about 5 mg/kg to about 50 mg/kg, from about 10 mg/kg to about 40 mg/kg, and from about 15 mg/kg to about 30 mg/kg. In another embodiment, the effective amount of the CXCL5 antagonist has a lower limit chosen from 0.01 mg/kg, 0.05 mg/kg, 0.1 mg/kg, 0.5 mg/kg, 1 mg/kg, 5 mg/kg, 10 mg/kg, 15 mg/kg, 20 mg/kg and 25 mg/kg, and an upper limit chosen from 100 mg/kg, 90 mg/kg, 80 mg/kg, 70 mg/kg, 60 mg/kg, 50 mg/kg and 40 mg/kg.

In one embodiment of the present disclosure, the CXCL5 antagonist is administered 1 to 2 times over a period of 2 to 4 days. In another embodiment, the CXCL5 antagonist is administered 8 to 15 times over a period of 3 to 5 weeks. For example, the CXCL5 antagonist is administered 3 times over a week, or 10 times over a period of 4 weeks. In yet another embodiment, the CXCL5 antagonist is administered 24 hours apart.

As used herein, the term “administering” or “administration” refers to the placement of an active agent (e.g., CXCL5 antagonist) into a subject by a method or route which results in at least partial localization of the active agent at a desired site such that a desired effect is produced. The active agent described herein can be administered by any appropriate route known in the art including, but not limited to, oral or parenteral routes, including intraperitoneal, intravenous, intradermal, intramuscular, subcutaneous, or transdermal routes.

In one embodiment of the present disclosure, the CXCL5 antagonist may be presented in a pharmaceutical composition to be administered to the subject. In certain embodiments, the present disclosure provides a pharmaceutical composition for stimulating angiogenesis, comprising the CXCL5 antagonist and a pharmaceutically acceptable carrier. The pharmaceutical composition provided in the present disclosure may efficiently prevent or treat PAOD and/or a peripheral ischemic tissue or a tissue damaged by peripheral ischemia.

In one embodiment of the present disclosure, the pharmaceutically acceptable carrier may be a diluent, a disintegrant, a binder, a lubricant, a glidant, a surfactant, or a combination thereof.

In one embodiment of the present disclosure, the pharmaceutical composition is a sterile injectable composition, which may be a solution or suspension in a non-toxic parenterally acceptable diluent or solvent. Among the acceptable vehicles and solvents that can be employed are 1,3-butanediol, mannitol, water, Ringer's solution, and isotonic sodium chloride solution. In addition, fixed oils are conventionally employed as a solvent or suspending medium (e.g., synthetic mono- or di-glycerides). Fatty acids, such as oleic acid and its glyceride derivatives, are useful in the preparation of injectables, as naturally pharmaceutically acceptable oils, such as olive oil and castor oil, especially in their polyoxyethylated versions. These oil solutions or suspensions can also contain a long chain alcohol diluent or dispersant, carboxymethyl cellulose, or similar dispersing agents. Other commonly used surfactants such as Tweens and Spans or other similar emulsifying agents or bioavailability enhancers which are commonly used in the manufacture of pharmaceutically acceptable solid, liquid, or other dosage forms can also be used for the purpose of formulation.

The carrier in the pharmaceutical composition must be “acceptable” in the sense that it is compatible with the active agent of the composition (and can be capable of stabilizing the active agent) and not deleterious to the subject to be treated. One or more solubilizing agents can be utilized as pharmaceutical excipients for delivery of an active compound. Examples of other carriers include colloidal silicon oxide, magnesium stearate, cellulose, and sodium lauryl sulfate.

Many examples have been used to illustrate the present disclosure. The examples below should not be taken as a limit to the scope of the present disclosure.

### EXAMPLES

### Example 1: Measurements of CXCL5 Levels in Plasma and Endothelial Progenitor Cells (EPCs)

For cell culture of EPCs, blood samples were obtained from the peripheral veins of healthy volunteers or patients with type 2 diabetes mellitus (DM) in the morning hours after an overnight fasting. In this study, only stable type 2 DM patients without insulin treatment were enrolled, and patients with other significant systemic diseases, receiving major operation in the past 6 months, or currently under medical treatment for other diseases were excluded. The human study was approved by the institute research committee and conformed with the Declaration of Helsinki.

After blood sampling, the total mononuclear cells were isolated by density gradient centrifugation with Histopaque-1077 (1.077 g/mL, Sigma-Aldrich). In brief, mononuclear cells were plated in endothelial cell growth basal medium-2 (EBM-2; Lonza), with supplements (hydrocortisone, human fibroblast growth factors, vascular endothelial growth factor, R3-insulin-like growth factor-1, ascorbic acid, human epidermal growth factor, gentamicin sulfate-amphotericin, and 20% fetal bovine serum) on fibronectin-coated 6-well plates. After 4 days of culture, the medium was changed and non-adherent cells were removed; attached early EPCs appeared elongated with spindle shapes. Late (outgrow) EPCs emerged 2 to 4 weeks after the start of the culture of mononuclear cells. The late EPCs exhibited a cobblestone morphology and monolayer growth pattern typical of mature endothelial cells at confluence. EPCs were grown in EBM-2 supplemented with fetal bovine serum (5% v/v final concentration) in an atmosphere of 95% air and 5% CO₂ at 37° C.

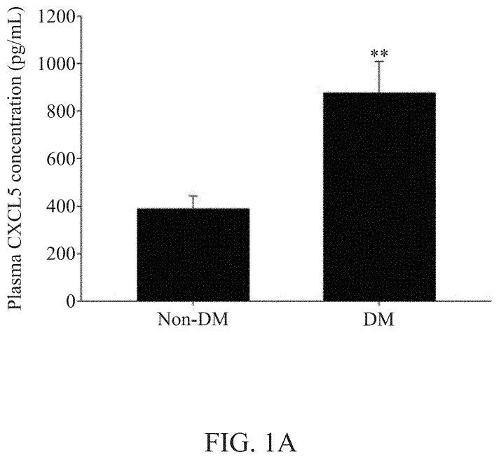

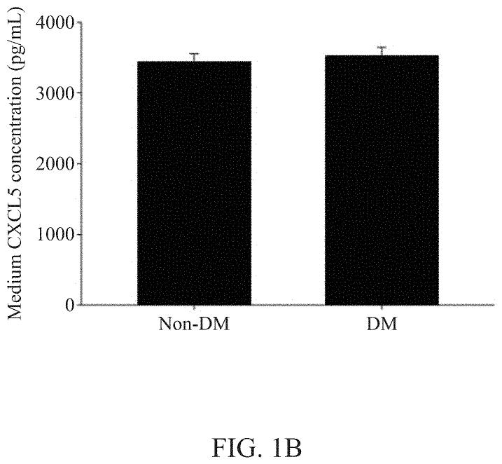

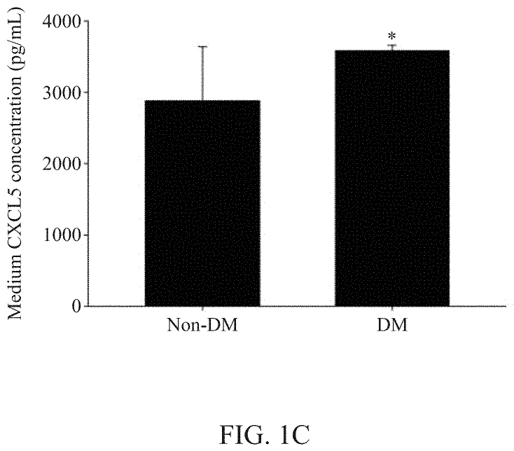

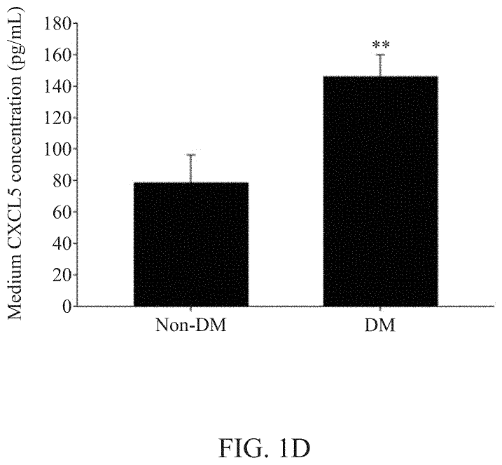

The levels of CXCL5 in plasma and in supernatants from mononuclear cells, early EPCs and late EPCs were measured by ELISA kits (R&D systems), according to the manufacturer's instructions. As shown in FIGS. 1A and 1B, the level of CXCL5 in plasma of the DM patients was higher than that of the healthy subjects (FIG. 1A), but there was no significant difference between the levels of CXCL5 in supernatants from mononuclear cells of the DM patients and the healthy subjects (FIG. 1B). Also, as shown in FIGS. 1C and 1D, it was found that the supernatants from early EPCs (FIG. 1C) and late EPCs (FIG. 1D) of the DM patients contain higher CXCL5 levels in comparison with the healthy subjects.

### Example 2: Effect of CXCL5 Neutralizing Antibody on the Functions of EPCs

EPCs were obtained by the process described in Example 1. Further, EPCs from healthy volunteers were cultured in high glucose (25 mM) for 3 days to obtain high glucose-stimulated EPCs. EPCs (1×10⁴ cells) from healthy volunteers and DM patients were individually resuspended in serum-free EBM-2 after pretreatment with CXCL5 monoclonal antibody (1 ng/mL or 10 ng/mL; R&D Systems) or IgG control for 24 hours for the subsequent migration assay and tube formation assay.

The migration of EPCs was evaluated using a chamber assay. The pretreated cells were added to the upper chambers of 24-well transwell plates with polycarbonate membranes. EBM-2 supplemented with 10% fetal bovine serum was added to the lower chamber. After incubation for 18 hours, the cells were fixed with 4% paraformaldehyde and stained using a hematoxylin solution. The numbers of migrated cells were counted in six random high-power (×100) microscopic fields.

For the tube formation assay, ECMatrix gel solution (Invitrogen) was mixed with ECMatrix diluent buffer (Invitrogen) and placed in a 96-well plate. Then, 1×10⁴ pretreated EPCs were placed in the matrix solution with EGM-2 supplemented with 10% fetal bovine serum and incubated for 16 hours in an atmosphere of 95% air and 5% CO₂ at 37° C. Tubule formation was inspected under an inverted light microscope (×40). Four representative fields were imaged, and the average of the total area of complete tubes formed by cells was compared using Image-Pro Plus software.

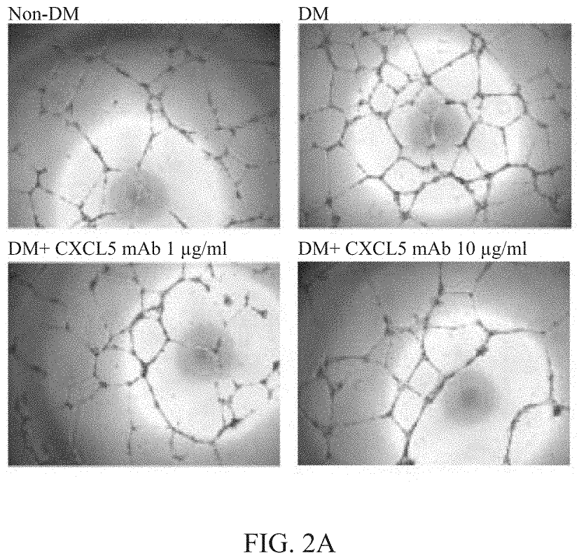

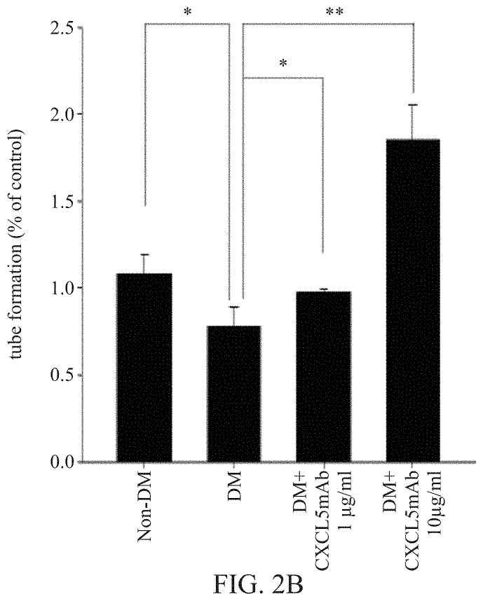

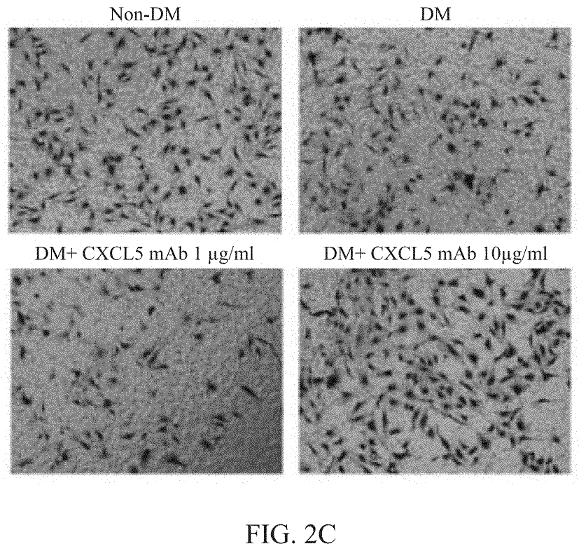

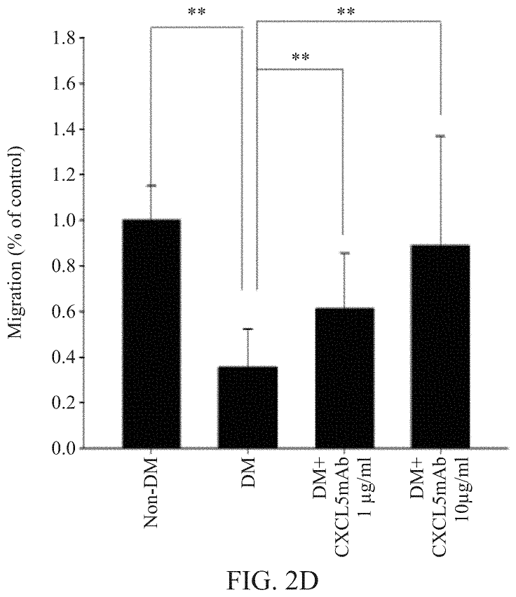

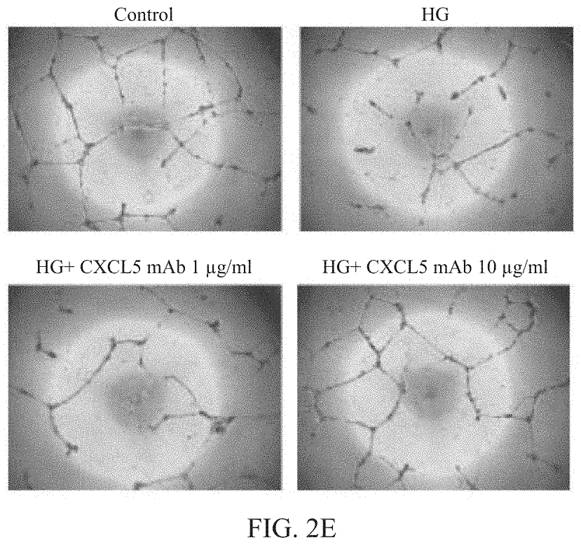

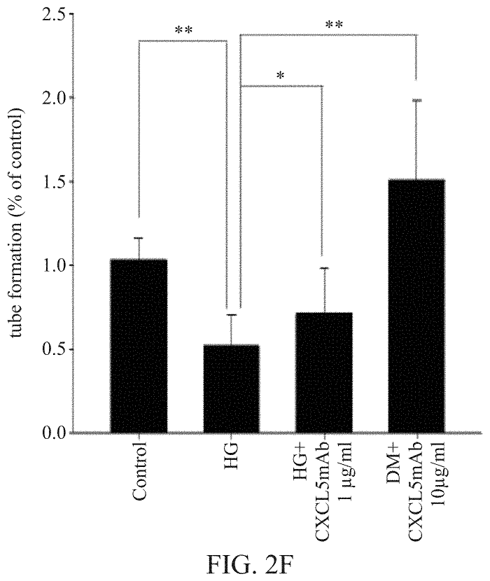

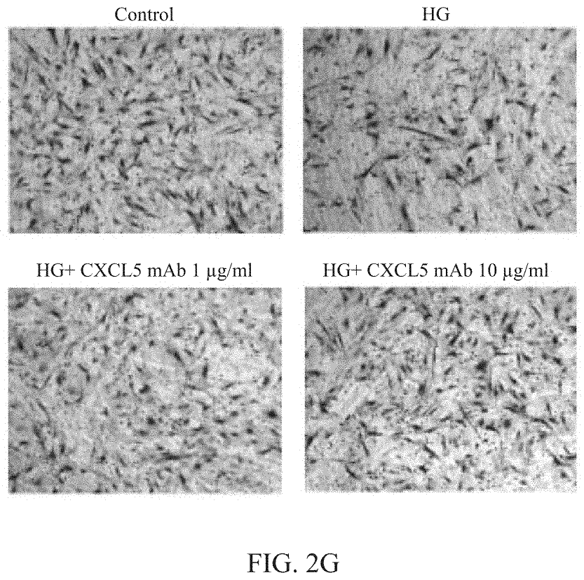

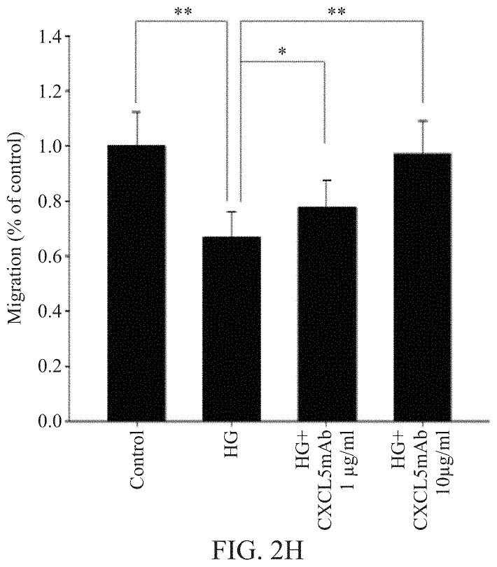

Referring to FIGS. 2A to 2D, it was observed that the abilities of tube formation and migration of EPCs from diabetic patients were improved by CXCL5 neutralizing antibody. Also, as shown in FIGS. 2E to 2H, CXCL5 neutralizing antibody improved the tube formation and migration of high glucose-stimulated EPCs from normal subjects. Accordingly, inhibition of CXCL5 may result in enhanced angiogenesis and migration of EPCs.

Western blot was further performed to detect the vascular endothelial growth factor (VEGF) and stromal cell-derived factor 1 (SDF-1) expressions in EPCs. Equal amounts of proteins obtained from EPCs were subjected to sodium dodecyl sulfate polyacrylamide gel electrophoresis (SDS-PAGE) using 8% to 12% gradient gels under reducing conditions (Bio-Rad Laboratories) and transferred to nitrocellulose membranes (Millipore). Membranes were incubated with antibodies against VEGF (Cell Signaling Technology), SDF-1 (Cell Signaling Technology), and actin (Cell Signaling Technology) at 4° C. overnight. The membranes were incubated with horseradish peroxidase (HRP)-conjugated secondary antibodies (1:1000) for 1 hour at room temperature. Finally, the membranes were visualized using an ECL kit (PerkinElmer).

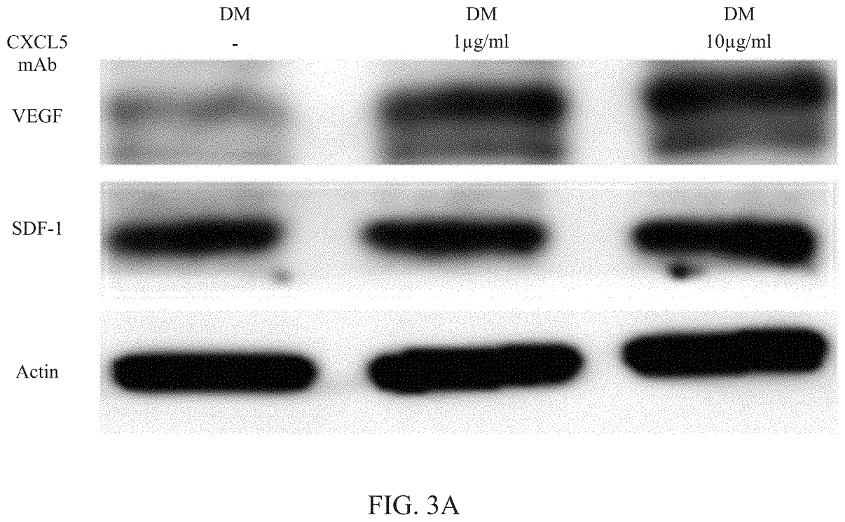

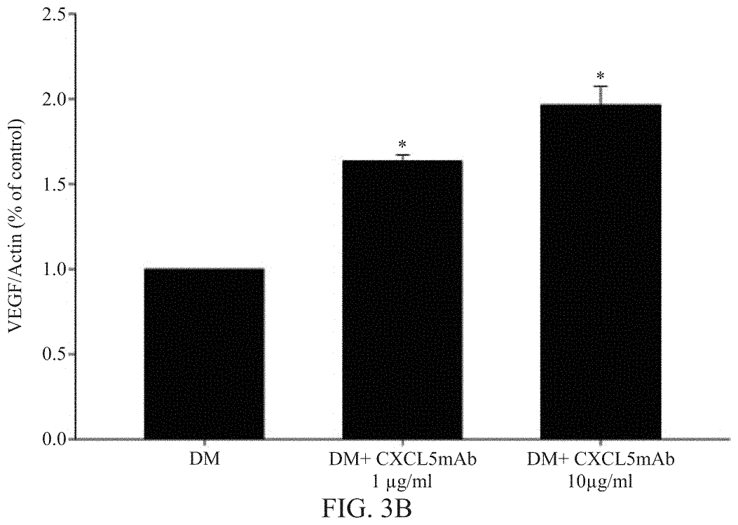

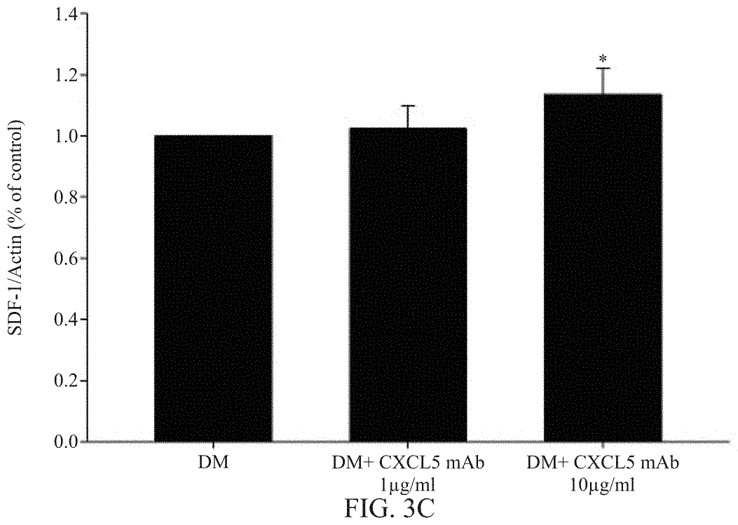

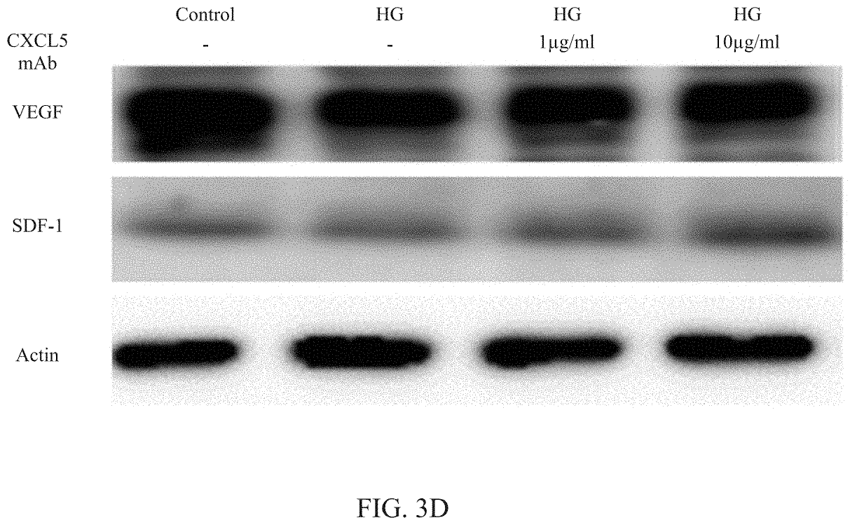

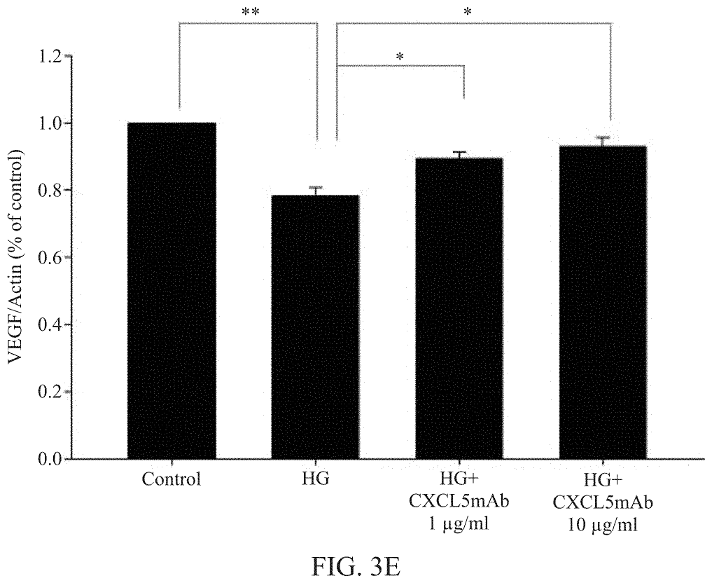

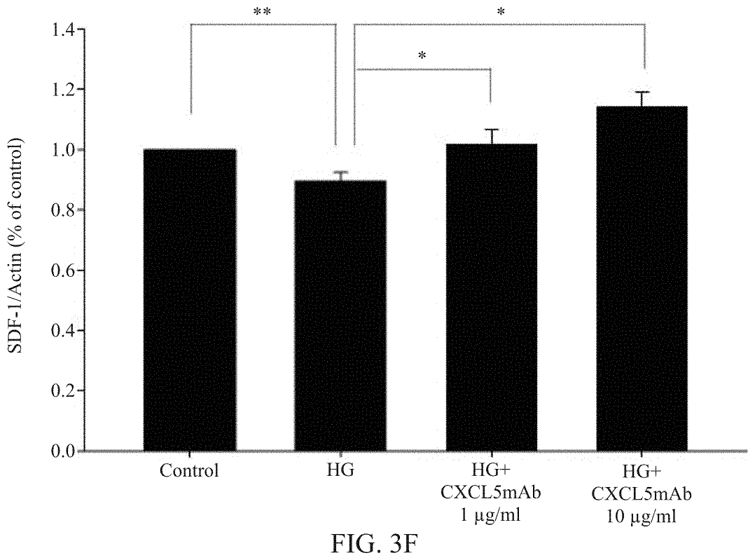

The results of Western blot and the statistical analyses were shown in FIGS. 3A to 3F. It was found that the VEGF and SDF-1 expressions in EPCs from diabetic patients or high glucose-stimulated EPCs from normal subjects were increased by the treatment with CXCL5 neutralizing antibody. These results indicated that CXCL5 neutralizing antibody improved angiogenesis and migration abilities of EPCs via VEGF and SDF-1 signaling pathways.

### Example 3: Effect of CXCL5 Neutralizing Antibody on Human Aortic Endothelial Cells (HAECs)

Primary HAECs (ScienCell, Carlsbad, Calif., USA) were cultured in fibronectin-coated plates with endothelial cell medium containing 5% fetal bovine serum, 1% endothelial cell growth supplement, and 1% penicillin/streptomycin solution at 37° C. in a humidified incubator with an atmosphere of 5% CO₂. The evaluations of the angiogenesis and migration abilities of HAECs and the VEGF and SDF-1 expressions in HAECs were performed according to the processes described in Example 2.

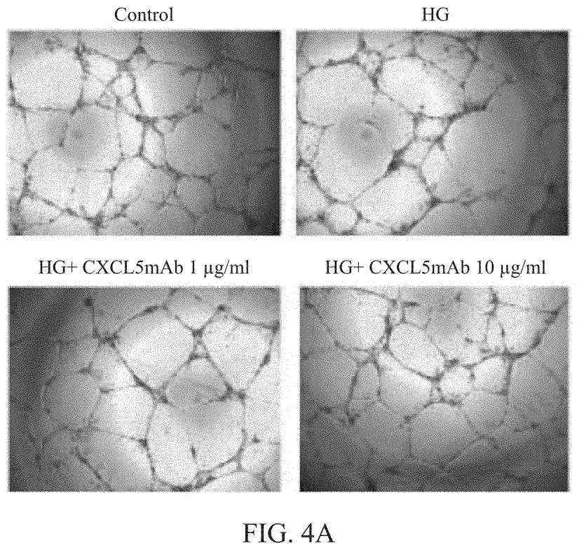

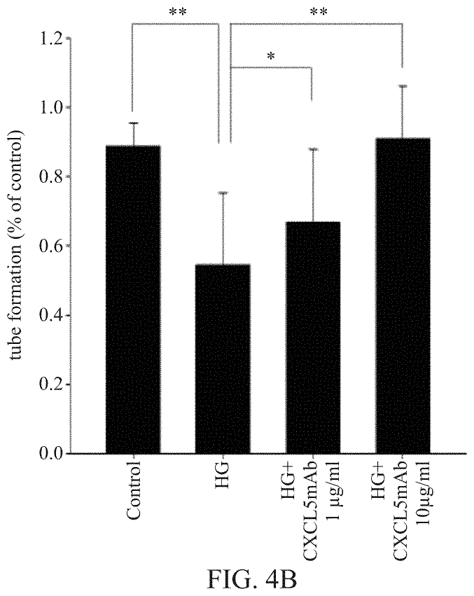

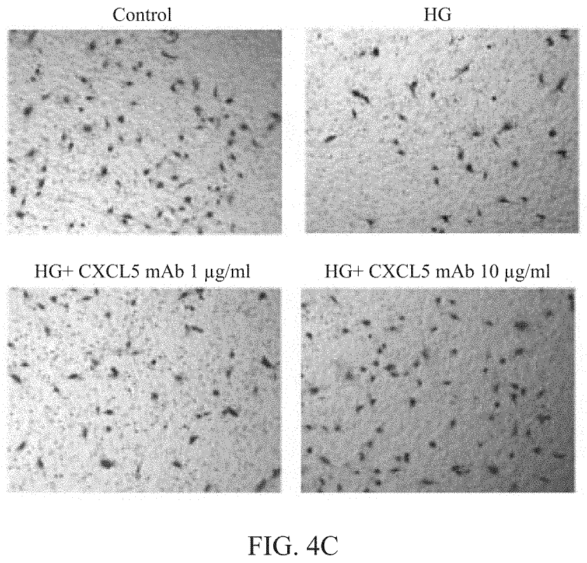

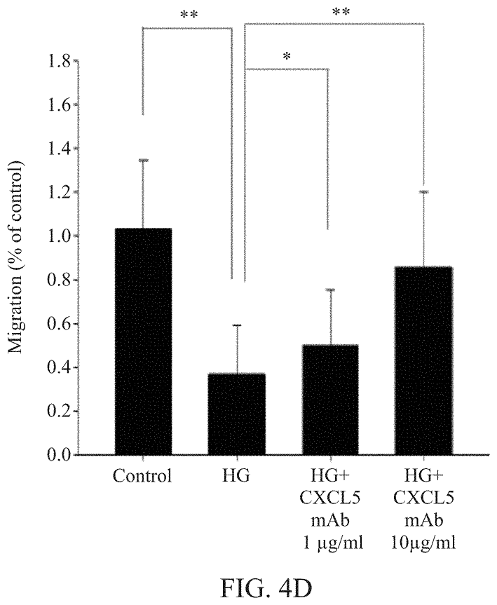

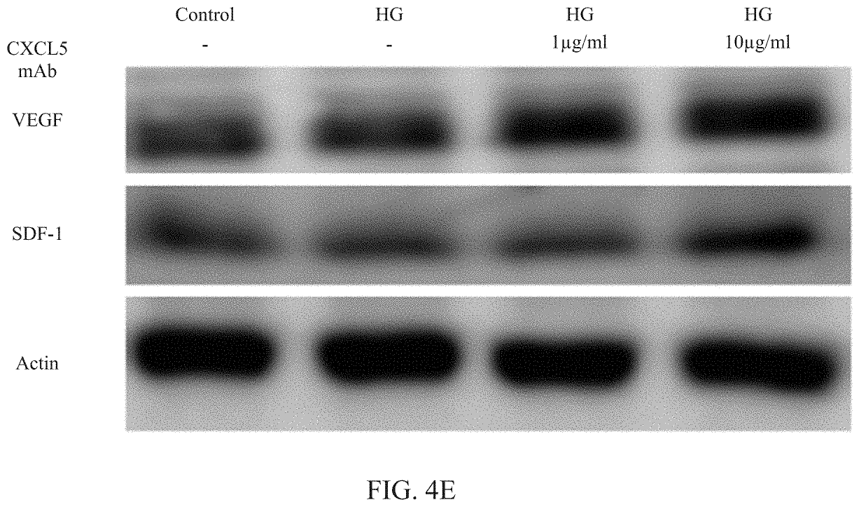

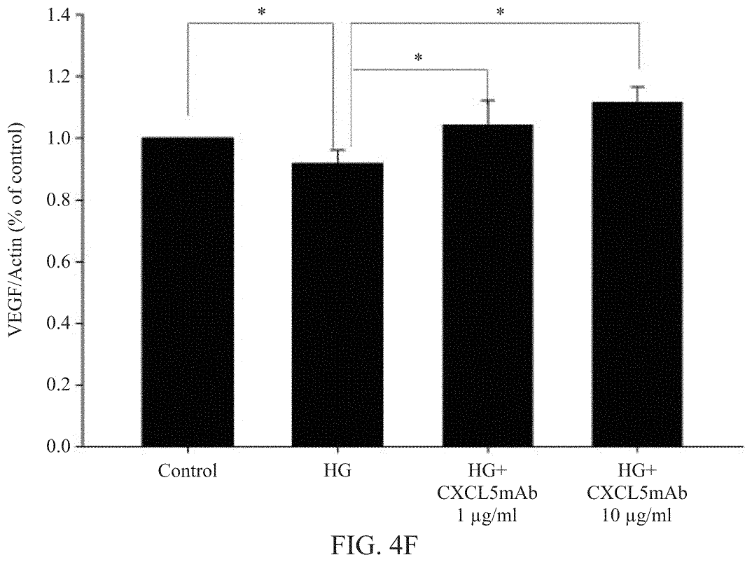

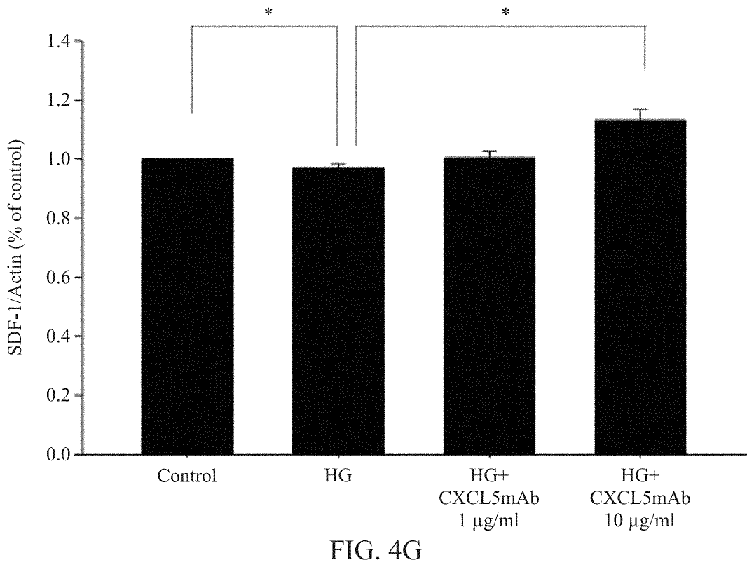

The results were shown in FIGS. 4A to 4G. It was observed that CXCL5 neutralizing antibody induced a significantly greater angiogenic response in the high glucose-stimulated HAECs by signaling through VEGF and SDF-1 pathways.

### Example 4: Animal Experiment and Establishment of Mouse Hindlimb Ischemia Model

To establish a diabetic mice model, six-week-old male FVB/NCrlBltw mice were purchased from BioLASCO (Taipei, Taiwan). FVB/NCrlBltw mice were acclimated for 2 weeks before being used to generate the type 1 diabetes mellitus (DM) model. To generate hyperglycemia in the FVB/NCrlBltw mice, FVB/NCrlBltw mice were intraperitoneally (i.p.) injected with streptozotocin (STZ) (40 mg/kg; Sigma-Aldrich) for 5 days.

Animals were raised according to the regulations of the Animal Care Committee of National Yang-Ming University. All animal-related work was performed under the Institutional Animal Care and Use Committee (IACUC) protocol approved by National Yang-Ming University.

For the treatment of CXCL5 antagonist, the diabetic mice received an intraperitoneal injection of an anti-CXCL5 neutralizing monoclonal antibody (10 μg or 100 μg; R&D Systems) immediately after the hindlimb ischemia surgery and 3 times per week for 4 weeks. The rat IgG2B isotype (10 μg or 100 μg; R&D Systems) was administered as the control.

The unilateral hindlimb ischemia was induced by excising the right femoral artery. Briefly, the animals in the individual group were first anaesthetized by i.p. injection of tribromothanol (240 mg/kg; Avertin; Sigma-Aldrich), and the proximal and distal portions of the right femoral artery and the distal portion of the right saphenous artery were ligated. The animals were sacrificed 4 weeks after the establishment of hindlimb ischemia.

### Example 5: Effect of CXCL5 Neutralizing Antibody on Angiogenesis in STZ-Induced Diabetic Mice

The animals with hindlimb ischemia descried in Example 4 were used in this example.

Hindlimb blood perfusion was measured with a laser Doppler perfusion imager system (Moor Instruments Limited) before, immediately after, and 4 weeks after the surgery. To avoid the influence of ambient temperature and the blood pressure changes of the animals, the results were expressed as the ratio of perfusion in the ischemic versus nonischemic limb.

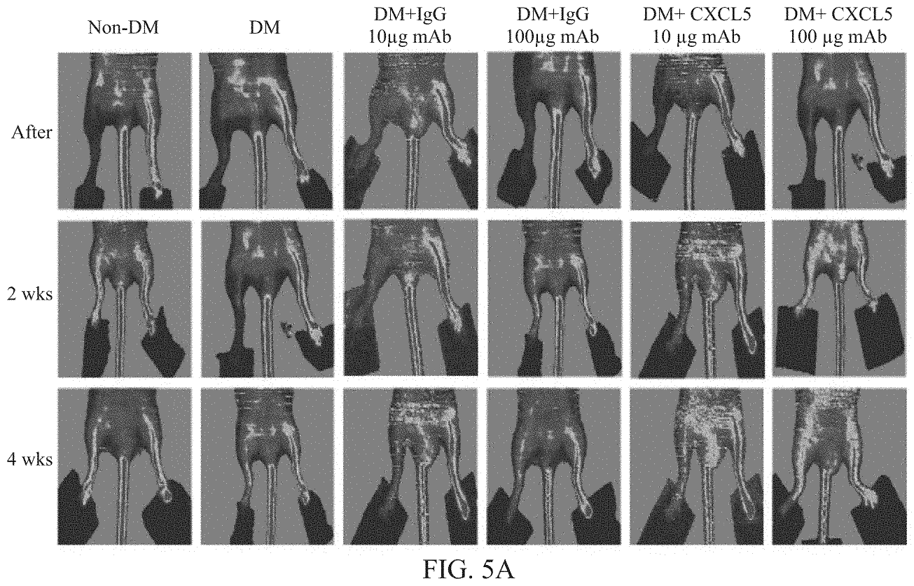

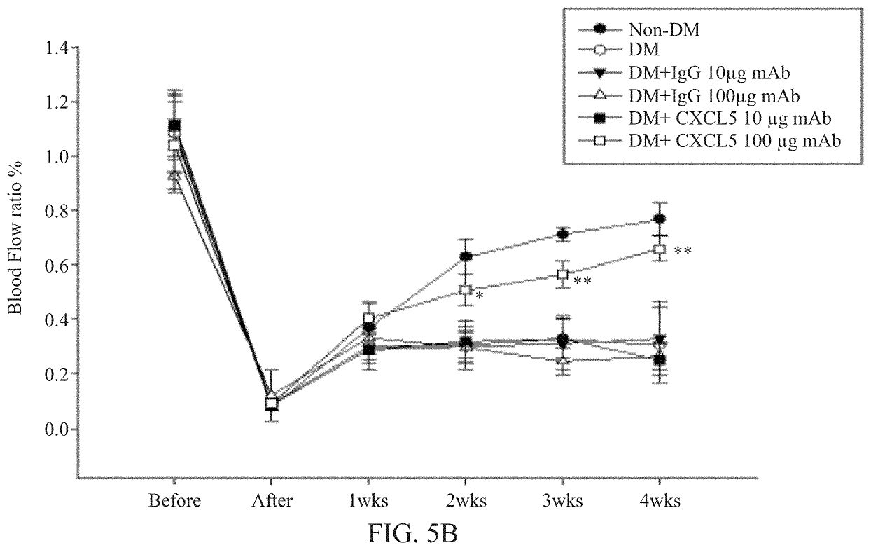

FIG. 5A showed the results of laser Doppler perfusion imaging. In the color-coded images, red indicates normal perfusion and blue indicates a marked reduction in blood flow in the ischemic hindlimb. FIG. 5B showed quantitative evaluation of blood flow expressed as a ratio of blood flow in ischemic limb to that in non-ischemic one. It was observed that hindlimb blood flow recovery occurred in non-DM animals and those treated with CXCL5 neutralizing antibody, indicating that CXCL5 antagonist was effective in improving ischemic tissues.

Further, for detecting the mobilization of EPC-like cells, peripheral blood samples were collected before and 2 days after the surgery, and the isolated mononuclear cells were incubated with fluorescein isothiocyanate (FITC) anti-mouse Sca-1 (eBioscience) and phycoerythrin anti-mouse Flk-1 (VEGFR-2, eBioscience) antibodies at 4° C. for 30 minutes. The expression of Sca-1⁺/Flk-1⁺ cells (i.e., EPC-like cells) in the mononuclear cells was analyzed by flow cytometry with a FACScalibur flow cytometer (BD Pharmingen). For analyses, 10⁵ circulating EPC-like cells were quantified by enumerating Sca-1⁺/Flk-1⁺ cells and were scored using FloJo (Treestar).

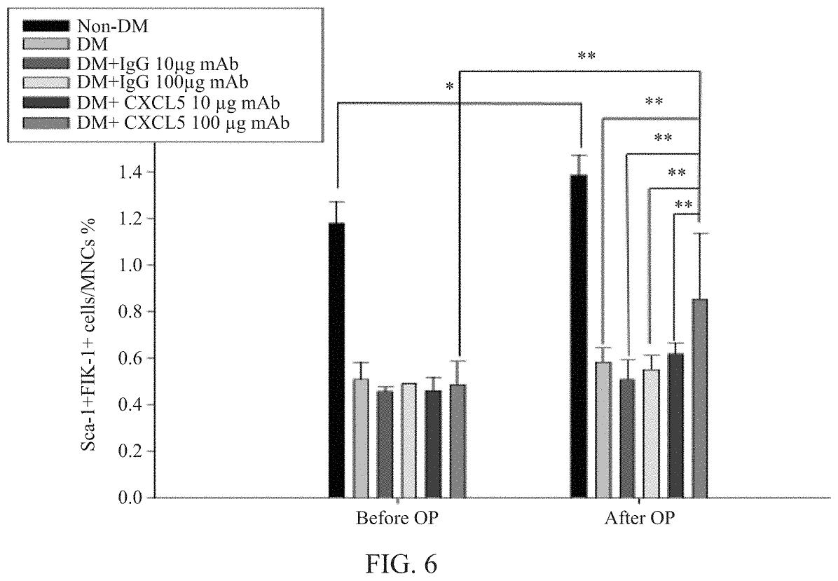

It has been reported that circulating EPCs are mobilized from bone marrow, and they play an important role in blood vessel repair and aid in reperfusion of the ischemic area. As shown in FIG. 6, it was found that the treatment of CXCL5 neutralizing antibody may rescue the decrease of circulating EPCs due to ischemia in the diabetic mice, implying that CXCL5 antagonist may involve in mobilizing or activating EPCs, and thus contributing to neovascularization.

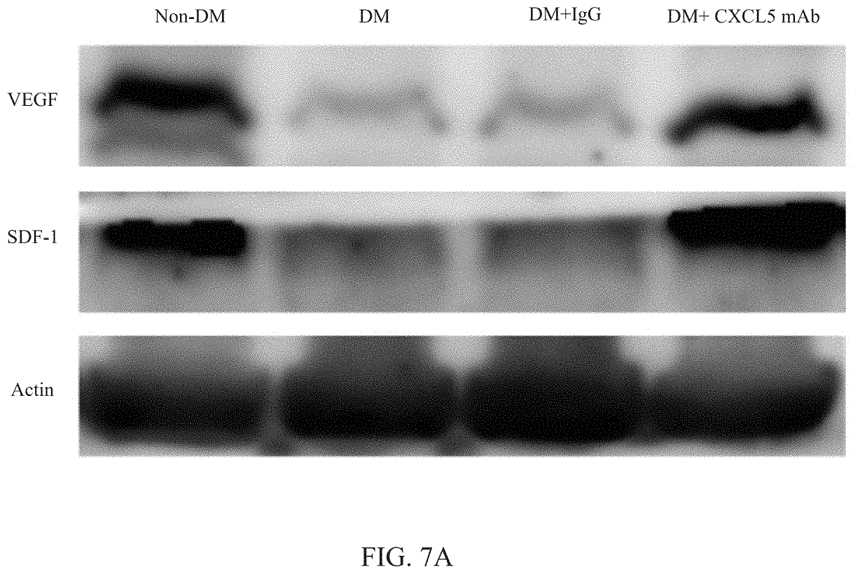

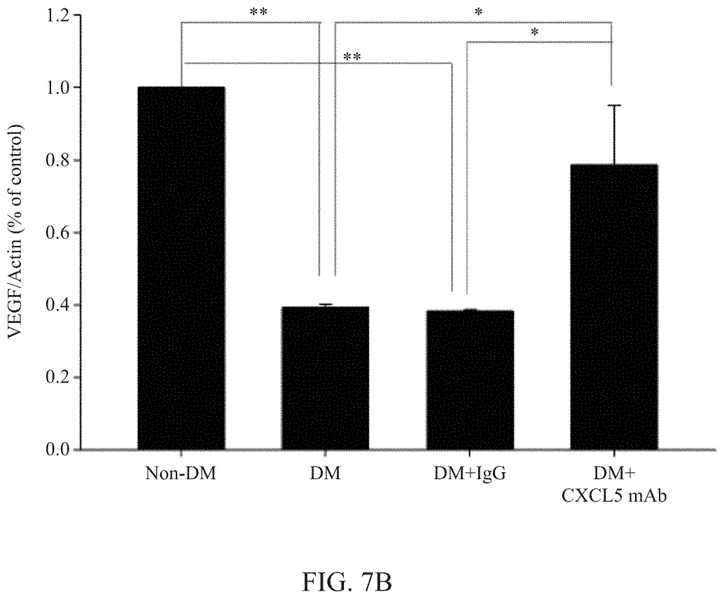

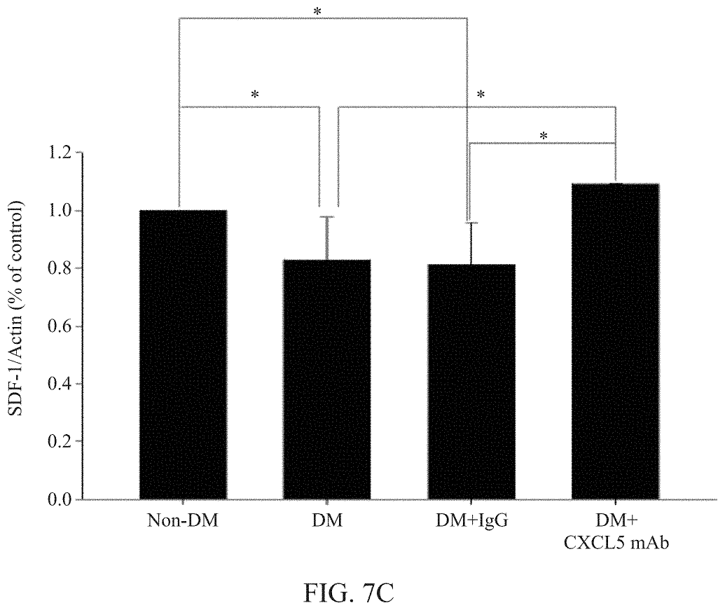

After the animals were sacrificed, the thigh muscles of mice were collected and used in Western blot to detect the VEGF and SDF-1 expressions. The results were shown in FIGS. 7A to 7C, indicating that the VEGF and SDF-1 expressions in diabetic mice were increased by the treatment with CXCL5 neutralizing antibody. It thus can be seen that CXCL5 neutralizing antibody may induce angiogenic response through VEGF and SDF-1 pathways.

### Example 6: Effect of CXCL5 Neutralizing Antibody on Wound Healing in STZ-Induced Diabetic Mice

The established diabetic mice and the drug-treatment protocol described in Example 4 were used in this example. For wound healing assay, mice were anesthetized, and the back skin was shaved and cleaned using an antibacterial soap solution and 75% alcohol. The circular full-thickness excisional wounds of 3 mm of diameter were generated with biopsy punch without injuring the muscle after CXCL5 antibodies were treated for 3 weeks. Wounds were recorded with a digital camera (Nikon).

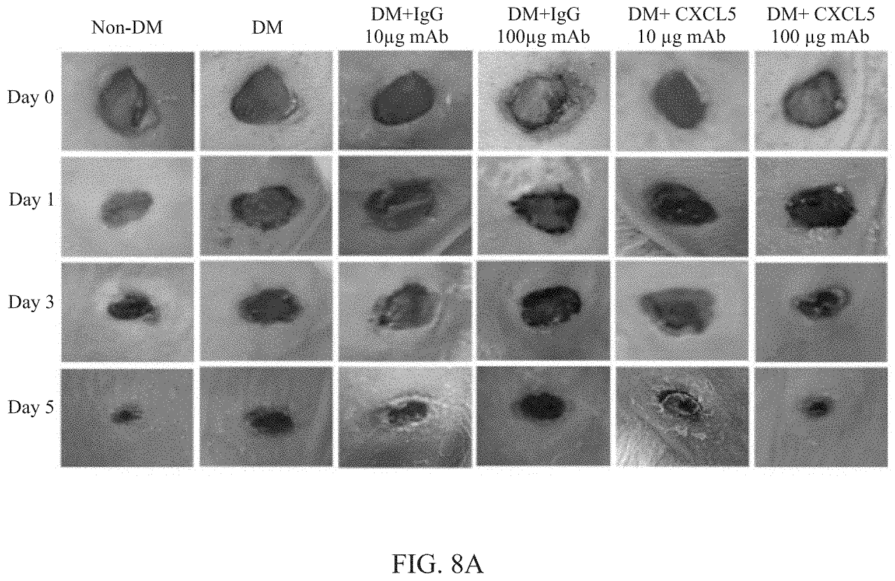

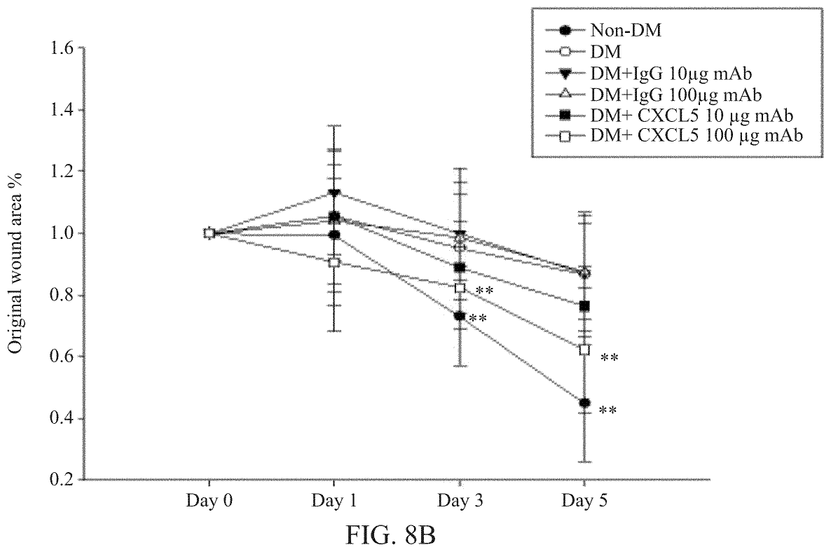

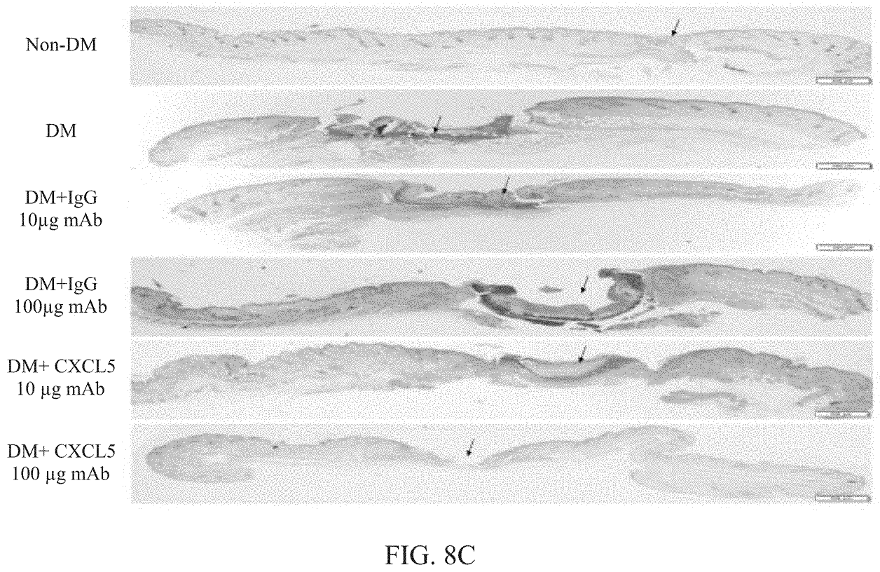

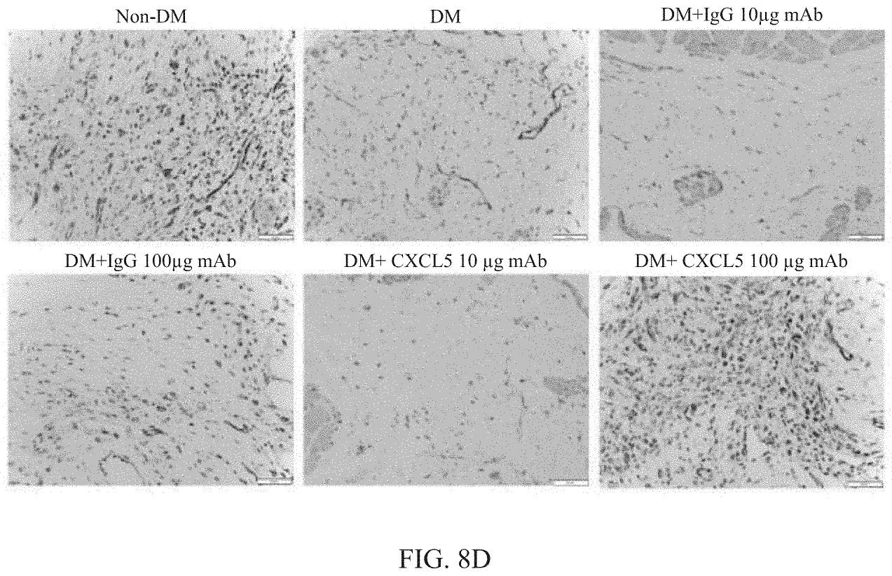

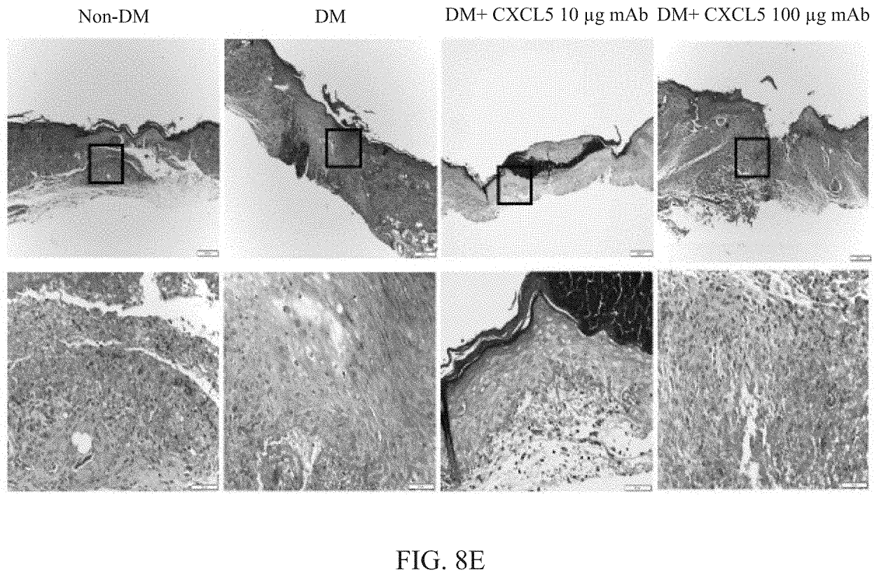

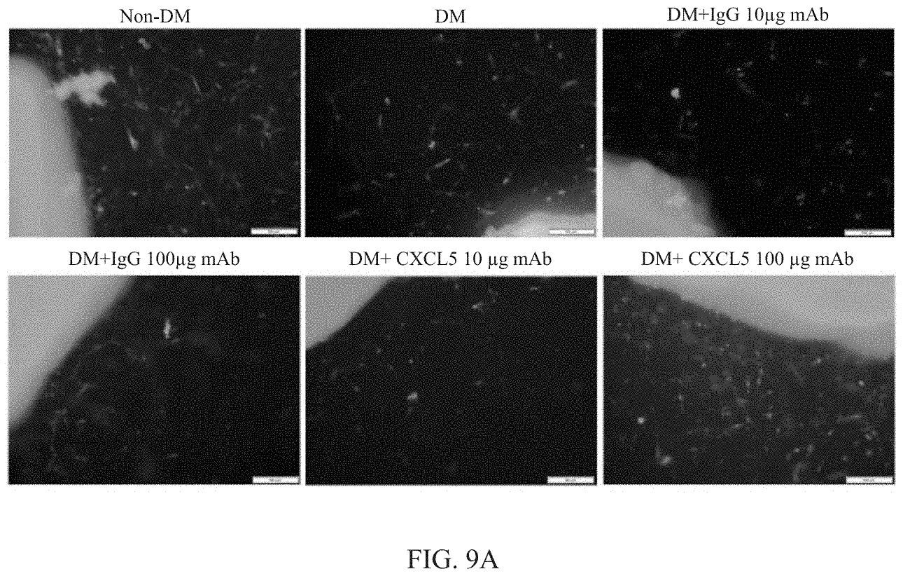

FIGS. 8A and 8B showed the representative photographs of the healing pattern of wounded skin of each group over a time period and the ratio of wound area (%) to the initial area on day 0 measured over time, respectively. These results suggested that the enhanced healing ability of wound was attributed to CXCL5 neutralizing antibody treatment.

At five days after injury, the animals were sacrificed, and the wounded skins were sampled for histological and immunohistochemistry analysis. The wound sample was fixed with 4% paraformaldehyde for 24 hours and dehydrated in graded alcohols. Then, the sample was embedded in paraffin wax and sectioned with a thickness of 5 μm. Some sections were de-paraffinized and incubated with H&E and Masson's trichrome stains for histological analysis. The other sections were de-paraffinized and incubated with a polyclonal rabbit anti-murine CD3.1 antibody (Abcam) at 4° C. overnight, followed by incubation with a secondary goat anti-rabbit antibody (Abcam).

FIG. 8C showed the H&E-stained sections. A significant increase in re-epithelialization was observed in the CXCL5 neutralizing antibody-treated group relative to the untreated diabetic group or the IgG-treated group.

FIG. 8D showed the anti-CD3.1 immunostaining sections. CD3.1, also known as platelet endothelial cell adhesion molecule-1 (PECAM-1), is a cell-surface receptor expressed on the membrane of endothelial cells, platelets, and most leukocyte subpopulations, and has been well defined as a marker of angiogenesis. As shown in FIG. 8D, the CD31-positive area was increased in the CXCL5 neutralizing antibody-treated group relative to the untreated control group or the IgG-treated group, indicating that CXCL5 neutralizing antibody may improve the development of angiogenic vessels.

FIG. 8E showed the wound collagen deposition performed by Masson's trichrome staining. Collagen synthesis plays a critical role in the process of skin wound healing, as it provides scaffolds for wound-healing cells and regenerative blood vessels, thereby promoting wound healing. Referring to FIG. 8E, more nascent collagen fibers were observed in the skin wound granulation tissue of the CXCL5 neutralizing antibody-treated group, while fewer collagen fibers were observed in the untreated control group.

The above results indicated that the CXCL5 neutralizing antibody may promote the development of angiogenic vessels and the deposition of wound collagen fibers, so as to accelerate skin wound healing.

### Example 7: Effect of CXCL5 Neutralizing Antibody on Aortic Sprouting Ex Vivo and Matrigel Plug Neovascularization In Vivo in STZ-Induced Diabetic Mice

The established diabetic mice and the drug-treatment protocol described in Example 4 were used in this example.

For aortic ring assay, mice were sacrificed with tribromothanol (240 mg/kg; Avertin; Sigma-Aldrich) after CXCL5 neutralizing antibodies were treated for 4 weeks and thoracic aortas were removed. The tissue was trimmed, and the blood was rinsed in the lumen with saline. The aortic rings were cut into a 0.5 mm of the descending thoracic aortas length and embedded with 1 mg/mL type 1 rat tail collagen matrix (Sigma-Aldrich, 08115), followed by incubation for 1 hour at 37° C. Aortic rings were cultured in EBM-2 (Lonza) containing 2.5% bovine serum (Gibco), 50 U/mL penicillin, 0.5 mg/mL streptomycin (Sigma-Aldrich) and 30 ng/mL VEGF (PEPROTECH) in 24 wells for 7 days. Aortic rings were incubated at 4° C. overnight with fluorescein isothiocyanate (FITC) anti-lectin B4 (Sigma-Aldrich, L9006). Images were captured using a fluorescent microscope (×100).

For matrigel plug neovascularization assay, mice were injected subcutaneously with growth factor reduced (GFR) basement membrane matrix (Corning Matrigel) containing 30 ng/mL VEGF (Cayman) and 50 U heparin (Sigma-Aldrich) after CXCL5 neutralizing antibodies were treated for 2 weeks. Plugs were collected after 14 days and homogenized in 500 μL of cell lysis buffer and centrifuged at 6000 g at 4° C. for 60 minutes. Hemoglobin was detected at 400 nm wavelength by using a colorimetric assay (Sigma). Also, plugs were harvested for histological and immunohistochemistry analysis. The process of histological and immunohistochemistry analysis was the same as that described in Example 6.

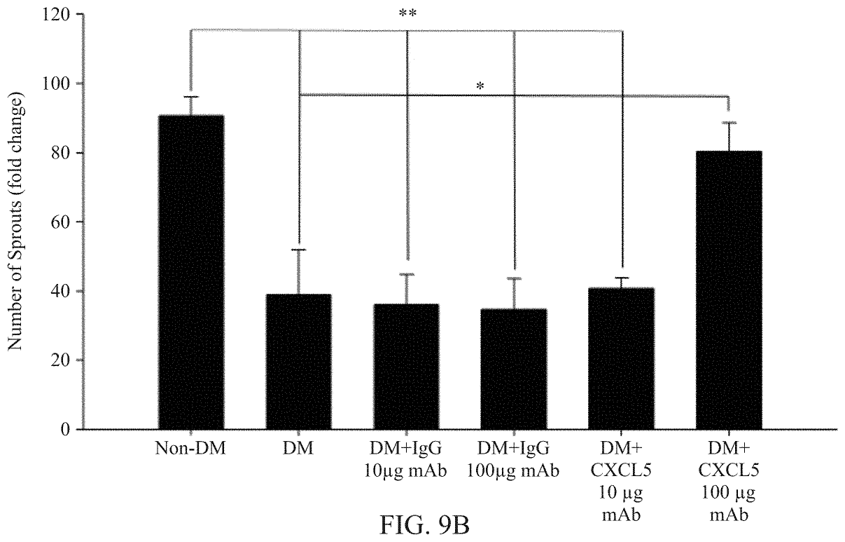

FIGS. 9A and 9B showed the vessel sprouting assay ex vivo and the quantitative analysis of lectin BS-I positive vessel sprouting number. Lectin BS-I is a specific marker for endothelial cells. These results indicated that CXCL5 neutralizing antibody may improve aortic sprouting, which representing the neovasculogenesis.

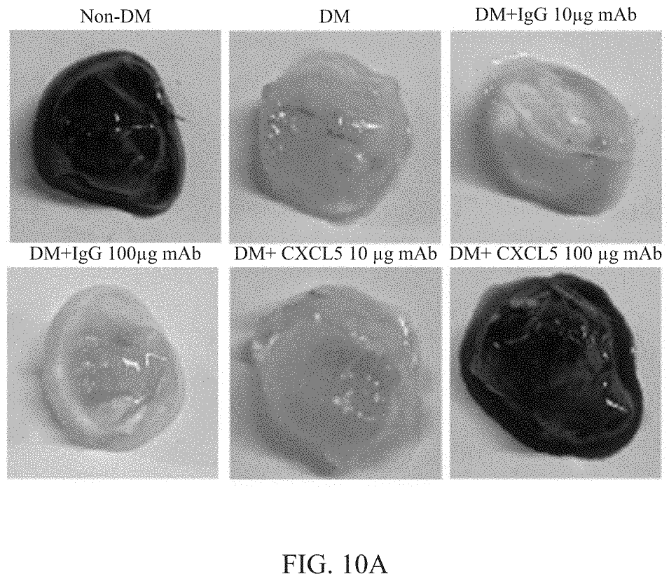

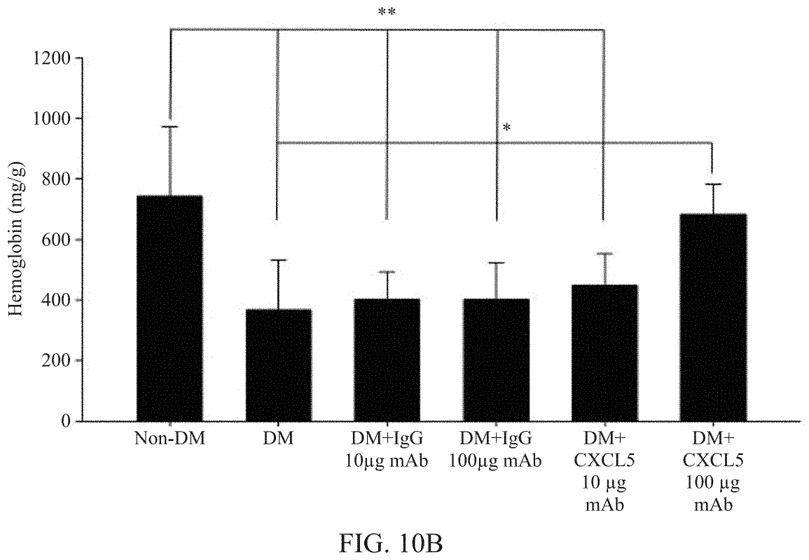

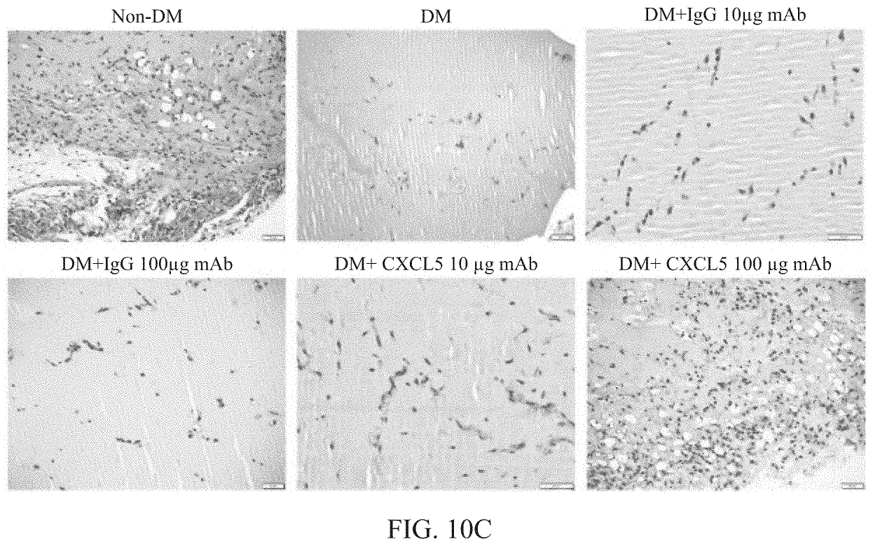

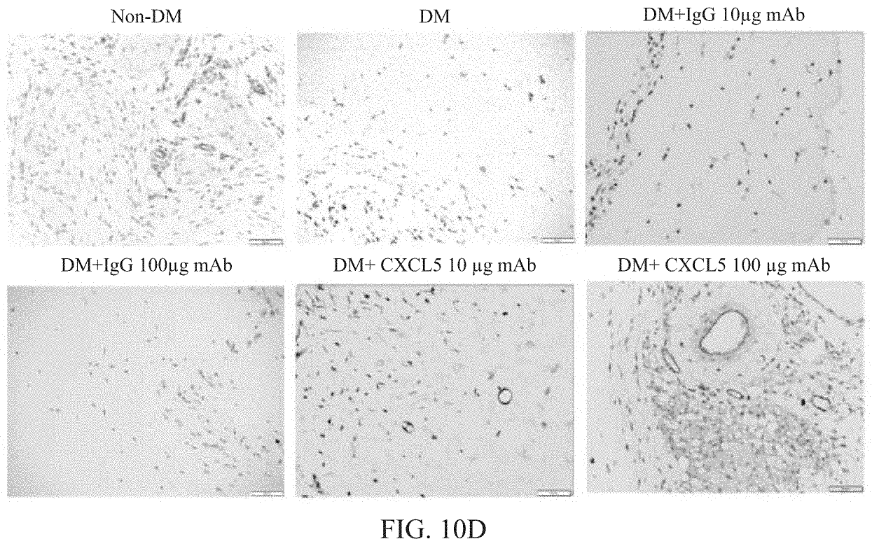

FIGS. 10A and 10B showed the matrigel plugs in the mice of each group and the quantitative analysis of neovascularization in matrigel plugs measured by hemoglobin contents. It was observed that CXCL5 neutralizing antibody improved the matrigel plug neovascularization in the diabetic mice. Further, FIGS. 10C and 10D showed the H&E-stained and anti-CD31 immunostaining sections of the matrigel plugs, demonstrating that CXCL5 neutralizing antibody increased neovasculogenesis.

### Statistics

The results shown in the above examples were given as the means±standard errors of the mean (SEM). Statistical analyses were performed using unpaired Student's t-test or analysis of variance, followed by Scheffe's multiple-comparison post hoc test. SPSS software (version 14; SPSS, Chicago, Ill., USA) was used to analyze the data. A p value of <0.05 was considered statistically significant.

From the above, the experiments indicate that the inhibition of CXCL5 may improve the functions of EPCs of a subject having impaired wound healing, such as the abilities of tube formation and migration. Also, the inhibition of CXCL5 rescues the functions of EPCs or HAECs that are impaired due to high glucose stimulation. As improving the cell functions, the expressions of angiogenesis factors, such as VEGF and SDF-1, are increased, thereby enhancing angiogenesis in the subject. Moreover, the inhibition of CXCL5 may increase the number of EPCs, promote angiogenesis, as well as induce vessel sprouting and tubulogenesis and the deposition of collagen in skin tissue, thereby improving ischemia and wound healing. Therefore, the method of using a CXCL5 antagonist of the present discourse is useful in accelerating the angiogenic process, and thus effective in treating PAOD and improving the peripheral ischemic tissue or the tissue damaged by peripheral ischemia.

While some of the embodiments of the present disclosure have been described in detail above, it is, however, possible for those of ordinary skill in the art to make various modifications and changes to the embodiments shown without substantially departing from the teaching and advantages of the present disclosure. Such modifications and changes are encompassed in the spirit and scope of the present disclosure as set forth in the appended claims.

### REFERENCE

[1] Sundaram, K., et al., CXCL5 stimulation of RANK ligand expression in Paget's disease of bone. Lab Invest, 2013. 93(4): p. 472-9.
[2] Smith, E., et al., Duffy antigen receptor for chemokines and CXCL5 are essential for the recruitment of neutrophils in a multicellular model of rheumatoid arthritis synovium. Arthritis Rheum, 2008. 58(7): p. 1968-73.
[3] Gardner, L., et al., The human Duffy antigen binds selected inflammatory but not homeostatic chemokines. Biochem Biophys Res Commun, 2004. 321(2): p. 306-12.

What is claimed is:

1. A method for preventing or treating a peripheral arterial occlusive disease (PAOD) in a subject in need thereof, comprising administering to the subject an effective amount of a CXC chemokine ligand 5 (CXCL5) antagonist for inhibiting CXCL5 activity.

2. The method according to claim 1, wherein the CXCL5 antagonist is an antibody or an aptamer directed against CXCL5 or a receptor of CXCL5.

3. The method according to claim 2, wherein the CXCL5 antagonist is an anti-CXCL5 antibody or a fragment thereof, a soluble form of CXC chemokine receptor 2 (CXCR2), a CXCR2 blocker, a soluble form of CXCR1, a CXCR1 blocker, a soluble form of Duffy antigen receptor for chemokine (DARC), or a DARC blocker.

4. The method according to claim 1, wherein the CXCL5 antagonist is selected from the group consisting of CXCL5 neutralizing antibody, AZD5069, reparixin, SB225002, SB265610, and a combination thereof.

5. The method according to claim 1, wherein the PAOD is limb ischemia, diabetic ulcer, gangrene, intermittent claudication, Buerger's syndrome, Raynaud's syndrome, or vasculitis.

6. The method according to claim 1, wherein the subject is directed to a human or animal that suffers from diabetes, chronic artery occlusion, vascular spasm, scleroderma, or vasculitis.

7. The method according to claim 1, wherein the effective amount of the CXCL5 antagonist is from about 0.01 mg/kg to about 100 mg/kg.

8. The method according to claim 7, wherein the effective amount of the CXCL5 antagonist is from about 0.1 mg/kg to about 80 mg/kg.

9. The method according to claim 1, wherein the CXCL5 antagonist is administered orally, intraperitoneally, intravenously, intradermally, intramuscularly, subcutaneously, or transdermally.

10. A method for preventing or treating a peripheral ischemic tissue or a tissue damaged by peripheral ischemia in a subject in need thereof, comprising administering to the subject a pharmaceutical composition comprising a CXC chemokine ligand 5 (CXCL5) antagonist in an effective amount to induce angiogenesis in the subject, and a pharmaceutically acceptable carrier thereof.

11. The method according to claim 10, wherein the CXCL5 antagonist is an anti-CXCL5 antibody or a fragment thereof, a soluble form of CXC chemokine receptor 2 (CXCR2), a CXCR2 blocker, a soluble form of CXCR1, a CXCR1 blocker, a soluble form of Duffy antigen receptor for chemokine (DARC), or a DARC blocker.

12. The method according to claim 10, wherein the peripheral ischemic tissue or the peripheral ischemia is caused by at least one of diabetes, chronic artery occlusion, Buerger's disease, Raynaud's disease, vascular spasm, scleroderma, and vasculitis.

13. The method according to claim 10, wherein the peripheral ischemic tissue or the tissue damaged by peripheral ischemia comprises a chronic wound, a digital ischemic lesion, a digital ulcer, or a digital necrotic lesion.

14. The method according to claim 10, wherein the administering promotes at least one of tissue healing, re-epithelialization of the tissue, and matrix deposition in the tissue.

15. The method according to claim 10, wherein the administering reduces at least one of rest pain associated with the peripheral ischemic tissue or the peripheral ischemia in the subject and development of a new peripheral ischemic tissue in the subject.
